(19) United States
(12) Patent Application Publication
(10) Pub. No.: US 2020/0037650 A1
(43) Pub. Date: Feb. 6, 2020

(54) FOOD FOR ENHANCING MUCOSAL IMMUNITY

(71) Applicant: KENKOUKAZOKU CO., LTD., Kagoshima-shi, Kagoshima (JP)

(72) Inventor: Yasushi NAKASONE, Kagoshima-shi (JP)

(73) Assignee: KENKOUKAZOKU CO., LTD., Kagoshima-shi, Kagoshima (JP)

(21) Appl. No.: 16/341,710

(22) PCT Filed: Jun. 5, 2017
(86) PCT No.: PCT/JP2017/020800

§ 371 Date: Apr. 12, 2019

(30) Foreign Application Priority Data
Oct. 13, 2016 (JP) 2016-201452

(51) Int. Cl. A23L 33/105; A61K 36/8962; A23L 29/00

(52) U.S. Cl. CPC A23L 33/105; A23L 29/035; A61K 36/8962

(57) ABSTRACT

The present invention relates to a food for enhancing mucosal immunity comprising black vinegar garlic as an active ingredient.

### TECHNICAL FIELD

The present invention relates to a food for enhancing mucosal immunity.

### BACKGROUND ART

It is known that garlic (Allium sativum) has an effect of enhancing immune cell function such as increasing the activity of gamma delta T cells and NK cells (Non-Patent Document 1).

The production of black vinegar (Kurozu) started about 200 years ago in Fukuyama region of Kagoshima prefecture and the production has been carried out by a traditional production method including three processes of saccharification, alcohol fermentation and acetic acid fermentation of rice, rice-malt (Koji), and water in a pot. Black vinegar is believed to have lipid metabolism improving action, erythrocyte deformability improving action, antiallergic action, blood pressure regulating action, blood sugar regulating action and the like.

Black vinegar garlic is believed to have a a nutrition and revitalization effect as well as a fatigue recovery effect, and many supplements are commercially available. However, no reports have been provided regarding the specific immunomodulatory effect of black vinegar garlic supported by experimental data.

### PRIOR ART DOCUMENTS

### Non-Patent Documents

Non-Patent Document 1: Nantz MP et al, Clin Nutr 2012 31(3) 337-44

### SUMMARY OF INVENTION

### Technical Problem

The present invention provides a food for enhancing mucosal immunity.

### Means for Solving the Problems

As a result of dedicated research, the present inventors have surprisingly found that black vinegar garlic enhances the release of secretory immunoglobulin A in saliva, and thus completed the present invention. That is, the present invention is as follows:
[1] A food for enhancing mucosal immunity comprising black vinegar garlic as an active ingredient.
[2] The food according to [1], wherein the black vinegar is an organic germinated brown rice black vinegar.
[3] The food according to [1] or [2], wherein the garlic is an organic garlic.
[4] The food according to [1] to [3], further comprising an organic black vinegar mash (moromi).

In the present invention, black vinegar garlic refers to those in which garlic is immersed in black vinegar. The immersion period is at least 2 weeks, preferably 3 months or more. In some cases, black vinegar garlic can further contain black vinegar mash (moromi). The entire black vinegar garlic can be made into a powder, and preferably, it can be freeze-dried.

In the present invention, conventionally known edible garlic can be used as a garlic. It is preferable that the garlic is a garlic cultivated by organic agriculture (also referred to as an organic garlic in the present invention). Organic agriculture refers to an agriculture carried out by using a method of agricultural production which has reduced environmental load deriving from agricultural production to the most possible extent, based on the non-use of chemically synthesized fertilizers and agrochemicals as well as the non-use of gene recombination technology. More specifically, those that were cultivated in the field where agrochemicals and chemical fertilizers have not been used for 3 years or more are referred to as organic agricultural products (Japanese Agriculture Standard for organic agricultural products).

In the present invention, conventionally known edible black vinegar can be used as a black vinegar; however, it is preferable that it is a rice black vinegar. Rice black vinegar refers to, among the grain vinegars, those only using rice (excluding those that have been milled by removing all of the bran layer of the brown rice, the same definition applies herein below) or wheat or barley added to this, as an ingredient, in which the amount of rice to be used is 180 g or more per 1 L of grain vinegar which is colored brown or dark brown by fermentation and aging (Vinegar quality labeling standards, Aug. 31, 2011, Consumer Affairs Agency Notice No. 8).

In the present invention, black vinegar mash (moromi) refers to a precipitate resulting from the process of preparing black vinegar.

The food of the present invention can be used in the form of soft capsules, tablets, hard capsules, powders, and granules.

As a function of the food of the present invention, for example, enhancement of mucosal immunity, prevention of disease, improvement of immunity, prevention of the upper respiratory tract infection, prevention of infection such as influenza and cold can be given.

### Effects of the Invention

The food of the present invention can enhance mucosal immunity.

### BRIEF DESCRIPTION OF THE DRAWINGS

FIG. 1: Time course of the change of the influence of black vinegar garlic ingestion on sIgA (secretory immunoglobulin A) release in saliva is shown. The vertical axis represents the change in the rate of sIgA release in saliva (μg/min). Placebo is group P, Active is group A.

Hereinafter, the present invention will be described in further details by way of examples; however, the present invention is not limited thereto.

### EXAMPLE 1

54 subjects were randomly divided into 2 groups; placebo food was administered to group P (28 subjects) and black vinegar garlic food was administered to group A (26 subjects). Each subject ingested 6 capsules per day. The composition per daily intake (6 capsules) is shown below.

| Component (mg) | Placebo | Black vinegar garlic |
|---|---|---|
| Flaxseed oil | 1,098 | 1,035 |
| Starch | 430.8 | 430.8 |
| Acetic acid flavor | 90.0 | 90.0 |
| Caramel color | 30.0 | 30.0 |
| Black vinegar garlic powder | 0.0 | 300.0 |
| Glycerol | 363.6 | 363.6 |
| Beeswax | 252.0 | 252.0 |
| Gelling agent | 165.6 | 165.6 |

Measurement of sIgA in Saliva

Before testing, the subjects rinsed their mouth twice and gargled twice with 150 ml of water. Fifteen minutes after being seated, the saliva was collected from the subjects for 120 seconds using Salisoft (Salisoft, Aarstedt, Numbrecht, Germany). The amount of the saliva collected was recorded. Concentration of sIgA in saliva was measured by immunoturbidimetric method according to BML's in-house protocol.

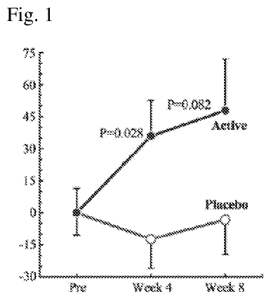

sIgA release rate in saliva was significantly higher in group A at week 4 than in group P (P=0.028) (FIG. 1). The rate in group A at week 8 was also higher than in group P (FIG. 1). The time course of change from the value before testing shows the increasing trend of sIgA secretion rate after ingestion of black vinegar garlic.

No adverse events were observed during the test.

From the above results, ingestion of food containing black vinegar garlic is believed to be beneficial in enhancing mucosal immunity.

sIgA constitutes the major mucosal immune effector molecule and provides an important front line for the defense against pathogens such as Streptococcus mutans and Porphyromonas gingivalis. It is hypothesized that lumen sIgA interferes with pathogens attached to mucosal epithelial cells and promotes intracellular neutralization of pathogens. IgA secreting cells differentiate in lymphoid tissues associated with mucosa. A majority of IgA secreting cells are present in the intestinal mucosa. Increasing evidence suggests that migration of antibody-secreting cells into the gut is important in conferring a protective immune response. The food of the present invention is provided as a capsule and the digestive tract is a tissue that is first exposed to the components contained in it. It is therefore considered that the immune system in the gastrointestinal tract was initially stimulated by black vinegar garlic and then it affected the sIgA release in saliva by the migration of the antibody-secreting cells to the oral mucosal tissue.

1. A method for enhancing mucosal immunity comprising the step of administrating black vinegar garlic to a subject in need thereof.

2. The method according to claim 1, wherein the black vinegar is an organic germinated brown rice black vinegar.

3. The method according to claim 2, wherein the garlic is an organic garlic.

4. The method according to claim 3, further administrating an organic black vinegar mash.
